(19) United States
(12) Patent Application Publication
(10) Pub. No.: US 2022/0122706 A1
(43) Pub. Date: Apr. 21, 2022

(54) UNIVERSAL VACCINATION ONLINE CERTIFICATE ISSUANCE SYSTEM

(71) Applicant: PROPHASE LABS, INC., Doylestown, PA (US)

(72) Inventor: Bedis Zormati, Merrick, NY (US)

(21) Appl. No.: 17/072,481

(22) Filed: Oct. 16, 2020

(51) Int. Cl. G16H 10/65; G16H 40/20; G06K 19/06; G16H 10/20; H04L 9/32

(52) U.S. Cl. CPC G16H 10/65; G16H 40/20; A61B 17/20; G16H 10/20; H04L 9/3263; G06K 19/06037

(57) ABSTRACT

A vaccination and immunization system designed to securely track administration of a vaccine for issuance of a related electronically-transmittable universal vaccination certificate for ensuring the safe and secure data collection of individuals being vaccinated and the reliable and trustworthy issuance of their resulting secured universal vaccine certificate.

### BACKGROUND OF THE INVENTION

The present invention relates to vaccination evidence and monitoring systems and, more particularly, a universal vaccination online certificate issuance and exchange system.

Vaccines typically require years of research and testing before reaching the clinical and regulatory approval, but scientists are racing to produce safe and effective vaccines. Once a vaccine is approved for use, researchers need to continue to monitor people who receive it to make sure the administration of the vaccination is consistently safe and effective.

Vaccine-monitoring systems currently in place will not work under the heavy demands necessary to properly respond to a pandemic or certain viruses requiring ongoing vaccinations, because such monitoring involves too many service providers and too many individuals from multiple jurisdictions and localities who cannot be tracked. Specifically, medical service providers are not able to collect data and keep track of individuals being vaccinated communicate to a centralized platform in real time, and once they are vaccinated the issuance of a certificate or legitimate proof may likely vary from various sources. Which leads to a central problem: once an individual has been vaccinated, how do we know he or she is immune and vaccinated and can no longer spread the virus and infect others?

No available software application enables technologically guide Medical Service Providers (MSPs) that will be administering the vaccine and enable MSPs to generate QR codes or alphanumeric pass codes which would translate into the issuance of an exportable, encrypted universal vaccination certificate that shall be on a standard recognizable format, with a predictable set of information.

As can be seen, there is a need for a universal vaccination online certificate issuance system that incorporates rules of a particular type and arrangement that improves the functions of the technological process of electronically issuing vaccination certification, a technological field vital for fighting contagions and pandemics. The system is colloquially known as “VaccTrack.”

In cryptography, a certification authority is an entity that issues digital certificate. A digital certificate certifies the ownership of a public key by the named subject of the certificate. This allows others (relying parties) to rely upon signatures or on assertions made about the private key that corresponds to the certified public key. The certification authority acts as a trusted third party—trusted both by the subject (owner) of the certificate and by the party relying upon the certificate.

The present invention will provide a cloud based universal platform for all relevant data pertaining to the individuals being vaccinated coupled to a certification authority configured to issue a universal secured, trusted encrypted vaccination certificate, thereby ensuring the safe and secure data collection of individuals being vaccinated.

The present invention may provide under one umbrella a cloud based database that will ensure MSPs proper and efficient administration of all vaccine recipients in a centralized universal database, whom will be issued a unique QR code and pass code which would be used to issue and generate an encrypted vaccination certificate.

### SUMMARY OF THE INVENTION

In one aspect of the present invention, a method for creating an electronically encrypted and exportable vaccination certificate, the method includes the following: validating, via a first processor, credentials of a medical service provider (MSP) through inputting one or more MSP data sets; collecting, via a second processor, one or more patient data sets; generating, via the second processor, an exportable identifier (EI); inputting, by the MSP through the first processor, a vaccination data set associated with the one or more patient data sets; and generating, by the second processor, an encrypted and exportable vaccination certificate (EEVC) when the second processor receives an indicator generated by the first processor of said inputting of the vaccination data.

In another aspect of the present invention, the method for creating an electronically encrypted and exportable vaccination certificate, the method includes the following: validating, via a first processor, credentials (using a verified NPI or DEA number of a medical service provider (MSP) through inputting one or more MSP data sets; collecting, via a second processor, one or more patient data sets; generating, via the second processor, a quick response code (QRC); inputting, by the MSP through the first processor, a vaccination data set associated with the one or more patient data sets, wherein the inputting of said vaccination data is conditioned of the MSP administrating a vaccine, wherein the vaccination data provides the following: at least one date of vaccination; at least one number of administrations of the vaccine; and the one or more MSP data sets; generating, by the second processor, an encrypted and exportable vaccination certificate (EEVC) when the second processor receives an indicator generated by the first processor of said inputting of the vaccination data, wherein the EEVC providing the following: the QCR; the vaccination data; and the one or more patient data sets; and representing, on the second processor a side effect tracker functionality configured to collect one or many self-reported adverse reactions to a vaccine. The system is colloquially known as “VaccWatch”.

These and other features, aspects and advantages of the present invention will become better understood with reference to the following drawings, description, and claims.

### BRIEF DESCRIPTION OF THE DRAWINGS

FIG. 1 is a schematic view of an exemplary embodiment of the present invention, illustrating a front page with sign in information such as log in button, verified email and password manual login, login with Facebook, Twitter, Google+, or the like; for both MSPs and the user/patients;

FIG. 2 is a schematic view of an exemplary embodiment of the present invention, illustrating a credential validation function for validating credentials for the Medical Service Provider (MSP) by using an NPI (National Provider Identifier) Number, which is a 10-digit numerical identifier, or a DEA number (DEA Registration Number), which is an identifier assigned to a health care provider (such as a physician, physician assistant, nurse practitioner, or the like;

FIG. 2A is a schematic view of an exemplary embodiment of the present invention, illustrating the MSP may either contact the user/patient or be contacted by the user/patient to schedule a vaccination appointment, which incorporating collection of information relevant to the administration of a vaccine, through generating and issuing a QR code or pass code upon vaccination completion;

FIG. 2B is a schematic view of an exemplary embodiment of the present invention, illustrating how a user may be prompted to upload an electronic HIPPA Form (e-HIPPA), which may be populated with the user information, and wherein the user may electronically sign the e-HIPPA form that shall be transmitted to anyone receiving the resulting immunization information, test results or any medical information subject to a HIPPA waiver, rendering the process a HIPPA compliant event. In the event the prospective user to be vaccinated is a minor, or an individual that requires a legal guardian or medical proxy, a sub-user certificate and functions shall be hosted within the main user's profile. Whether it is the parent, legal guardian or other authorized representative of the patient, the same benefits and functions of present invention may be granted, such as the ability to register, obtain, transmit to a third party the vaccination certificate, schedule appointments, self-report side effects using functionality disclosed herein all by the use of a signed HIPPA from the authorized representative;

FIG. 3 is a schematic view of an exemplary embodiment of the present invention, illustrating generation, transmission and/or prompting of a patient's unique QR code or (alphanumeric) passcode for smartphone, tablet, or PC;

FIG. 4 is a schematic view of an exemplary embodiment of the present invention, illustrating cloud database storage for vaccination data, user information, reported vaccine adverse reactions which may be stored on a secured cloud database to avoid duplication and conduct an efficient, streamlined vaccination campaign and provide real time vaccination campaign feedback to interested parties;

FIGS. 4A through 4C are schematic views of an exemplary embodiment of the present invention, illustrating MSP location functionality—e.g., use of IP or zip code based geo locator, search by name or an address which will display a list of MSPs offering the vaccination with the possibility to schedule an appointment and pick the day and time slot using the present invention's user interface;

FIG. 5 is a schematic view of an exemplary embodiment of the present invention, illustrating a scannable unique QR provided to the patient for creating a user profile using a camera of a smart phone or tablet. The QR code may also be provided by the MSP post vaccination;

FIG. 6 is a schematic view of an exemplary embodiment of the present invention, illustrating the user profile interface that can also be created on the application or PC using a unique passcode, with a verified email, a verified mobile phone number along with blood type rhesus, date of birth, city, state and country of vaccination, and/or the vaccination date (either pre-vaccination or post vaccination);

FIG. 7 is a schematic view of an exemplary embodiment of the present invention, illustrating the profile (after creation with the QR code or pass code) enabling the option to request an instant issue of an encrypted universal certificate, since upon completion of the vaccinating injection the QR code may be scanned or read with an iOS iPhone or Android phone that will issue an encrypted certificate that incorporates user information, such as the name, it will carry blood type rhesus, Date of Birth, City, State and Country of vaccination, and the Issue date or vaccination date, vaccine manufacturer, headshot (photo) or the user (for post-vaccination issue);

FIG. 8 is a schematic view of an exemplary embodiment of the present invention, illustrating the input of a unique passcode or scanning the QR code for electronically generating the exportable encrypted universal certificate;

FIG. 9 is a schematic view of an exemplary embodiment of the present invention, illustrating the exportable encrypted universal certificate on the user interface of a computing device, whereby the exportable encrypted universal certificate maybe converted to a PDF or JPEG file to be exported electronically, e.g., by email or text messaged, by fax, or the like;

FIGS. 10A through 10C are schematic views of an exemplary embodiment of the present invention, illustrating notification functionality enabling the user to setup alerts and reminders on a computer calendars, text message, phone calls and emails which could auto generate to remind the patient of the appointment, to get the vaccine, a reminder of the second dose of vaccine that must be injected within a certain time frame, and so on; and

FIGS. 11A through 11B are schematic views of an exemplary embodiment of the present invention, illustrating a side effect tracker functionality (colloquially known as “VaccWatch”): enabling injected/vaccinated recipients to self-report their side effects and detail any adverse reactions experienced in accordance with their race, gender, age group, ethnic background and the like, reporting serious reactions, product quality problems, therapeutic inequivalence/failure, and product use errors with human vaccination and immunization products, and the like.

In certain embodiments, injected patients who have experienced a serious reaction to a vaccine, may self-report on an anonymous basis any adverse reaction. VaccWatch may be able to immediately collect precise and real-time data. Subsequently the side effect tracker functionality may be able to analyze and compile reader friendly reports, charts to organize, and display side effects experienced by the vaccinated users in conjunctions to relevant factors such as their race, gender, age group, ethnic background, blood type rhesus, pre-existing conditions and locality. The side effect tracker functionality may provide a scroll down menu display of known side effects that users are able to select one or multiple side effect, users may also write in and describe other side effects not therein mentioned.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, the present invention embodies a vaccination and immunization software application designed to securely track administration of a vaccine for issuance of an electronically-transmittable universal vaccination certificate for ensuring the safe and secure data collection of individuals being vaccinated and the reliable and trustworthy issuance of the secured universal vaccine certificate.

In certain embodiments, the network may refer to any interconnecting system capable of transmitting audio, video, signals, data, messages, or any combination of the preceding. The network may include all or a portion of a public switched telephone network (PSTN), a public or private data network, a local area network (LAN), a metropolitan area network (MAN), a wide area network (WAN), a local, regional, or global communication or computer network such as the Internet, a wireline or wireless network, an enterprise intranet, or any other suitable communication link, including combinations thereof.

The server and the computer of the present invention may each include computing systems. The server and the computer may be embodied in a “processor”. This disclosure contemplates any suitable number of computing systems. This disclosure contemplates the computing system taking any suitable physical form. As example and not by way of limitation, the computing system may be a virtual machine (VM), an embedded computing system, a system-on-chip (SOC), a single-board computing system (SBC) (e.g., a computer-on-module (COM) or system-on-module (SOM)), a desktop computing system, a laptop or notebook computing system, a smart phone, an interactive kiosk, a mainframe, a mesh of computing systems, a server, an application server, or a combination of two or more of these. Where appropriate, the computing systems may include one or more computing systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing systems may perform at different times or at different locations one or more steps of one or more methods described or illustrated herein, where appropriate.

In some embodiments, the computing systems may execute any suitable operating system such as IBM's zSeries/Operating System (z/OS), MS-DOS, PC-DOS, MAC-OS, WINDOWS, UNIX, OpenVMS, an operating system based on LINUX, or any other appropriate operating system, including future operating systems. In some embodiments, the computing systems may be a web server running web server applications such as Apache, Microsoft's Internet Information Server™, and the like.

In particular embodiments, the computing systems includes a processor, a memory, a user interface, and a communication interface. In particular embodiments, the processor includes hardware for executing instructions, such as those making up a computer program. The memory includes main memory for storing instructions such as computer program(s) for the processor to execute, or data for processor to operate on. The memory may include mass storage for data and instructions such as the computer program. As an example and not by way of limitation, the memory may include an HDD, a floppy disk drive, flash memory, an optical disc, a magneto-optical disc, magnetic tape, a Universal Serial Bus (USB) drive, a solid-state drive (SSD), or a combination of two or more of these. The memory may include removable or non-removable (or fixed) media, where appropriate. The memory may be internal or external to computing system, where appropriate. In particular embodiments, the memory is non-volatile, solid-state memory.

The user interface includes hardware, software, or both providing one or more interfaces for communication between a person and the computer systems. As an example and not by way of limitation, an user interface device may include a keyboard, keypad, microphone, monitor, mouse, printer, scanner, speaker, still camera, stylus, tablet, touchscreen, trackball, video camera, another suitable user interface or a combination of two or more of these. A user interface may include one or more sensors. This disclosure contemplates any suitable user interface and any suitable user interfaces for them.

The communication interface includes hardware, software, or both providing one or more interfaces for communication (e.g., packet-based communication) between the computing systems over the network. As an example and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface. As an example and not by way of limitation, the computing systems may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computing systems may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The computing systems may include any suitable communication interface for any of these networks, where appropriate.

The present invention embodies a vaccination and immunization application designed to securely track a patient's recommended shots of a vaccine in order to generate the issuance of an electronically exportable and encrypted universal vaccination certificate, thereby ensuring the safe and secure data collection of individuals being vaccinated, efficiently streamlining the greater vaccination campaign and issuing of a generally accepted secured universal vaccine certificate.

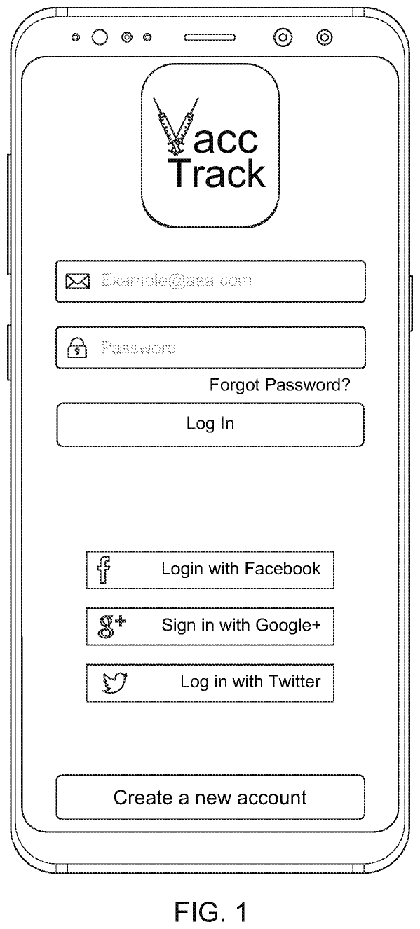

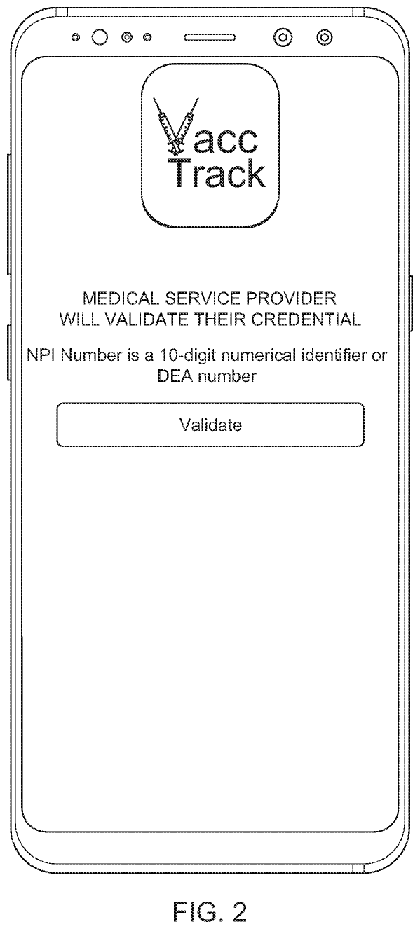

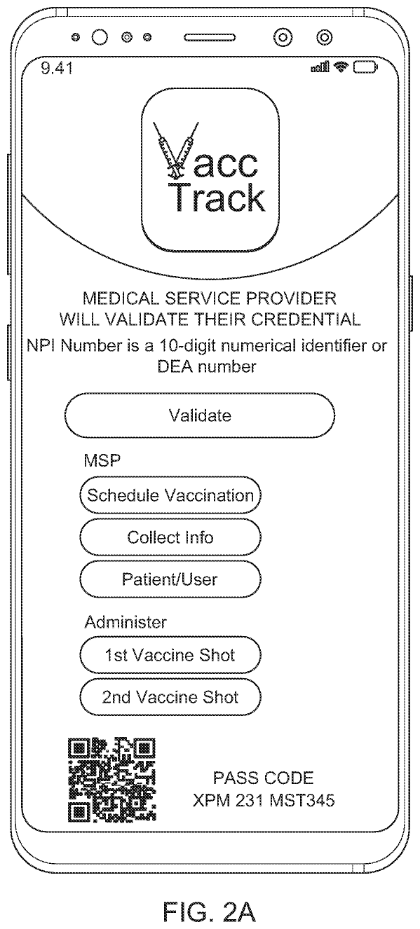

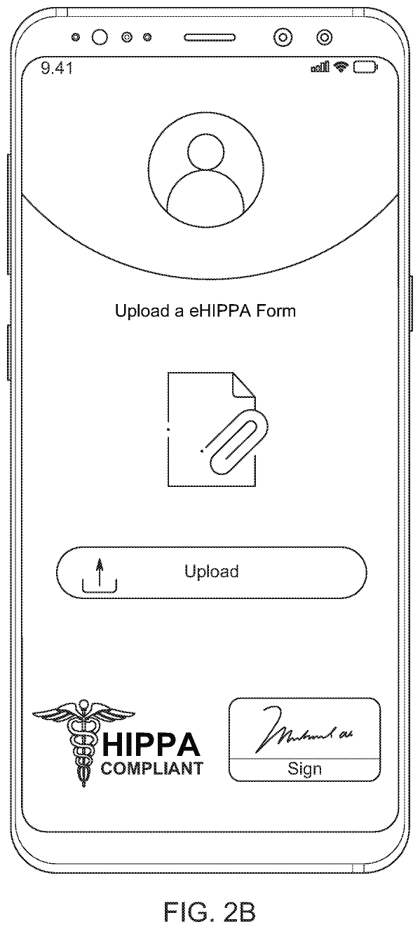

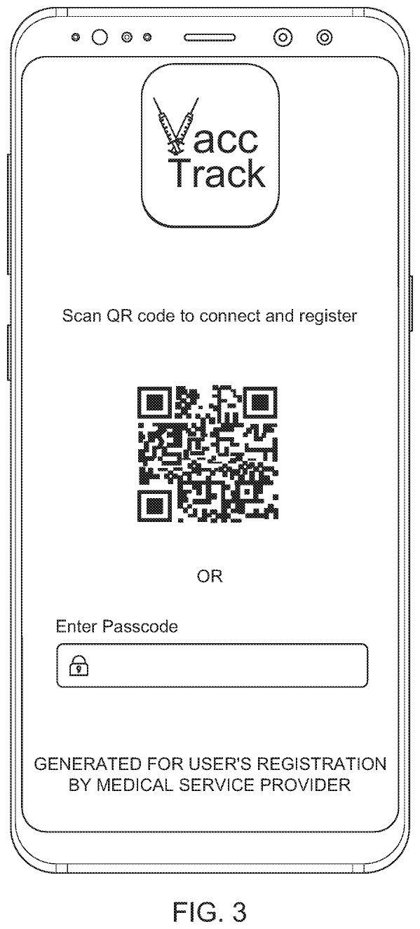

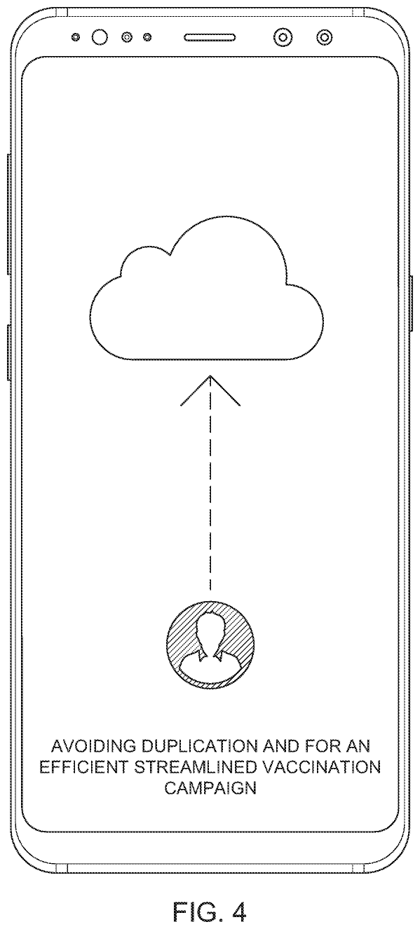

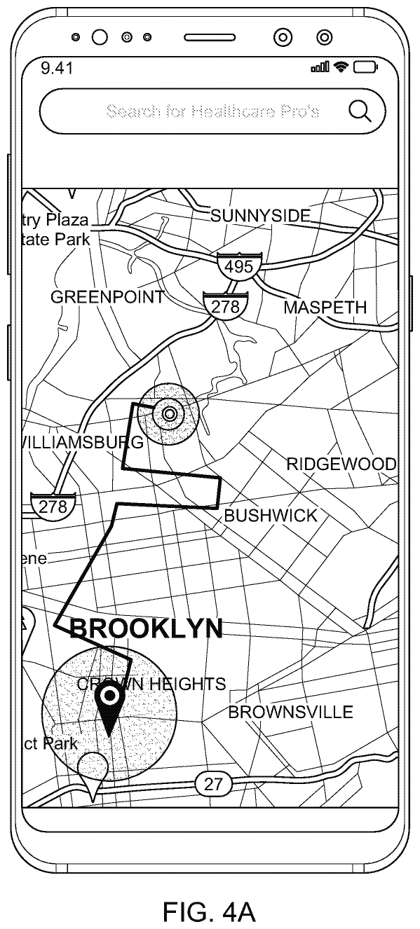

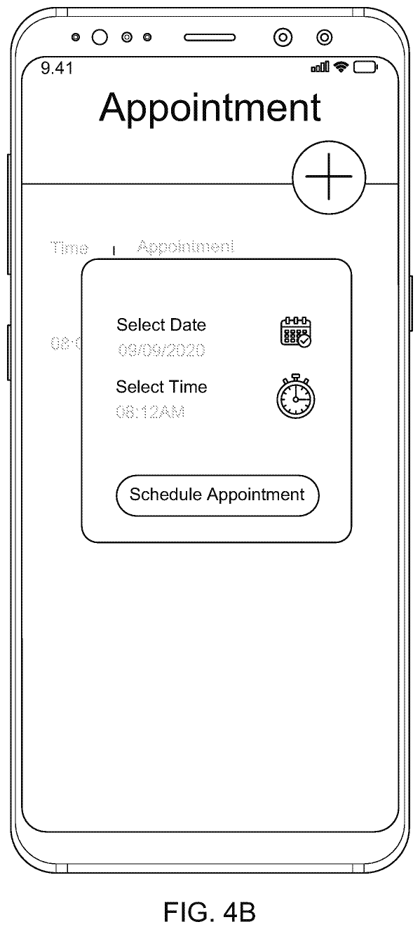

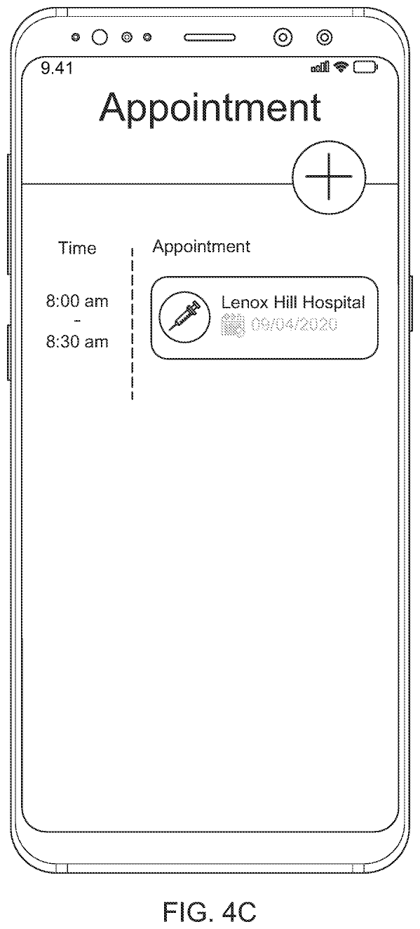

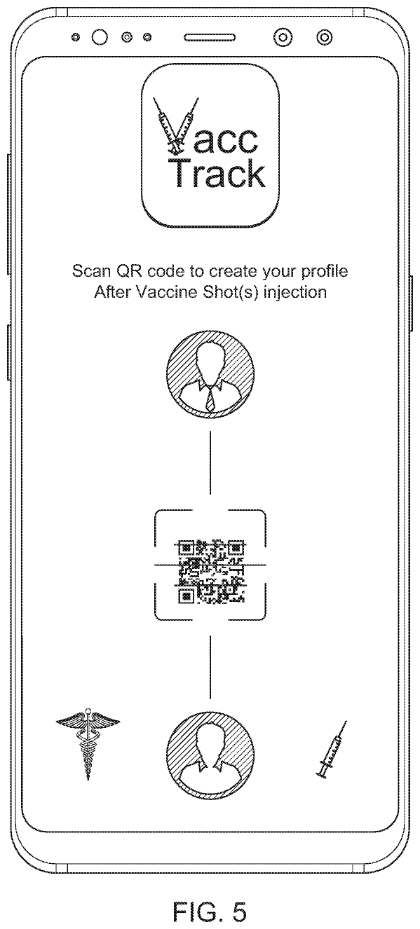

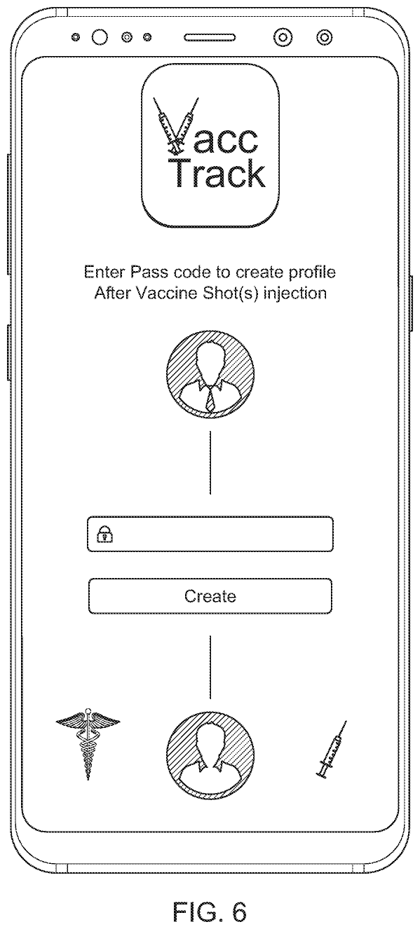

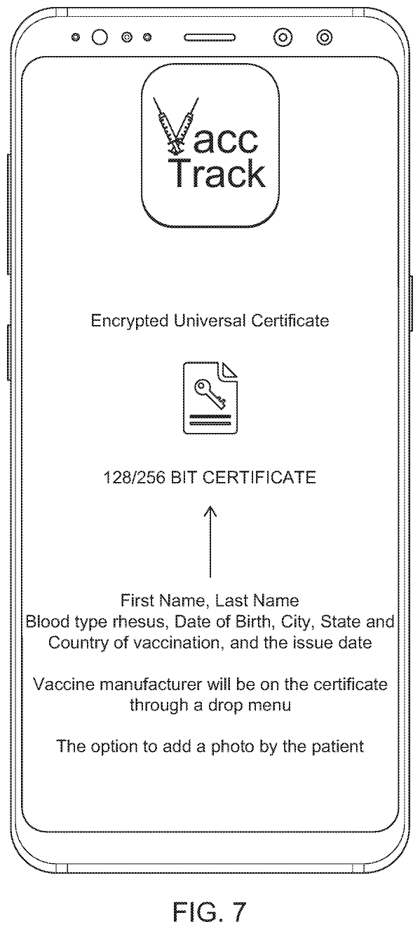

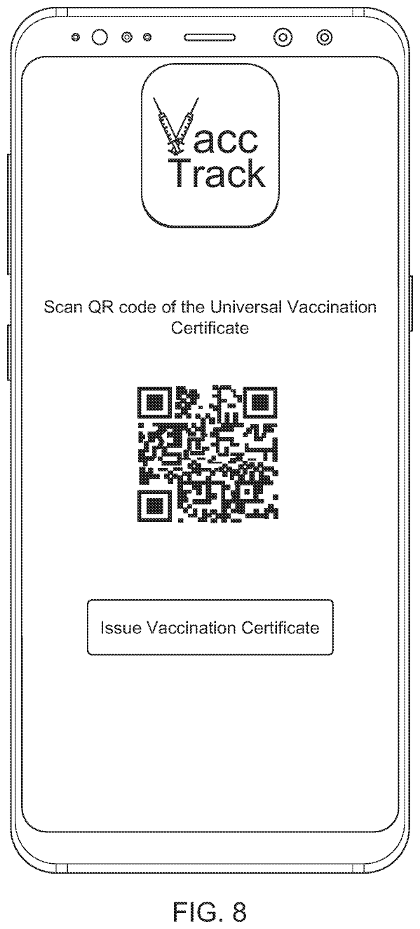

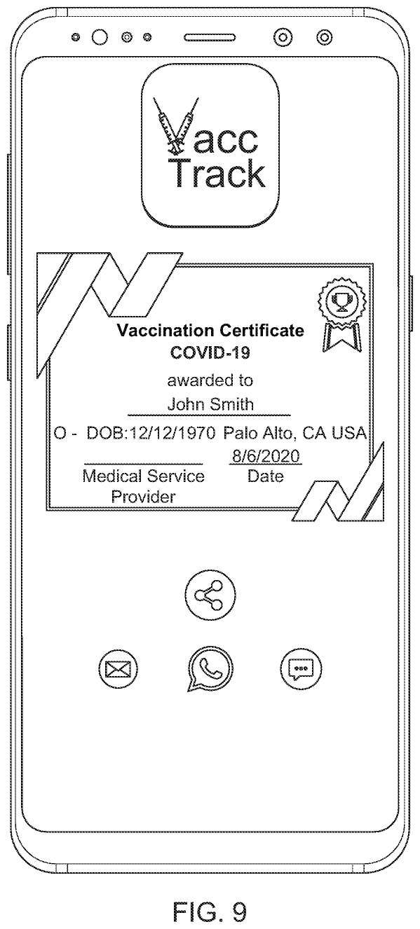

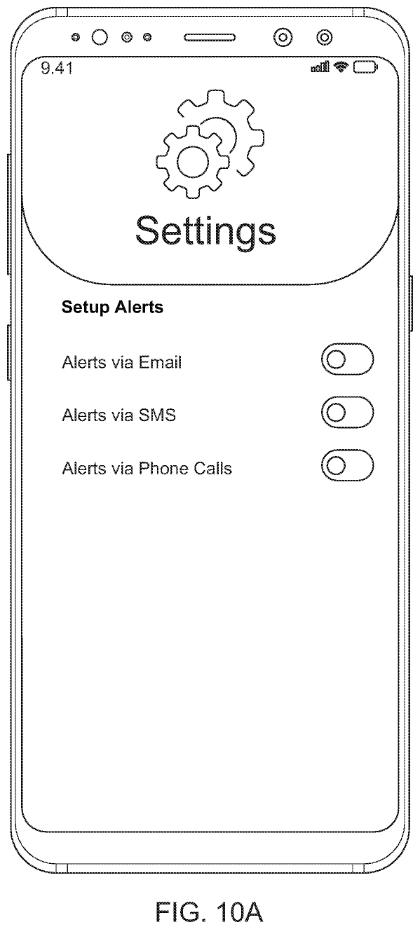

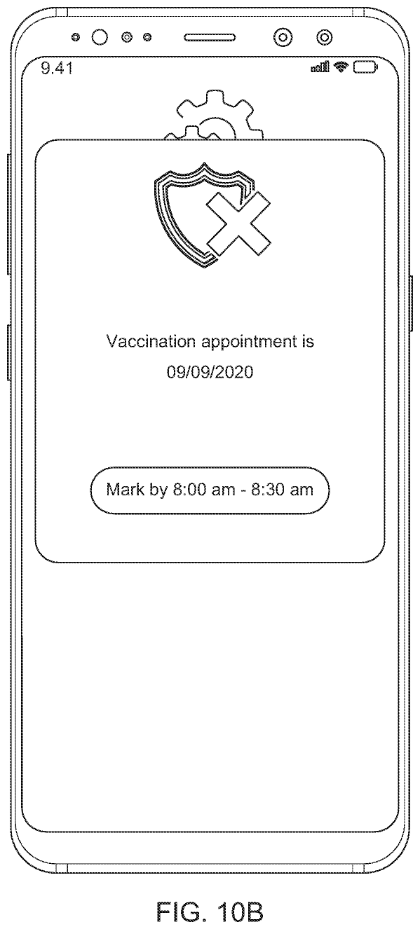

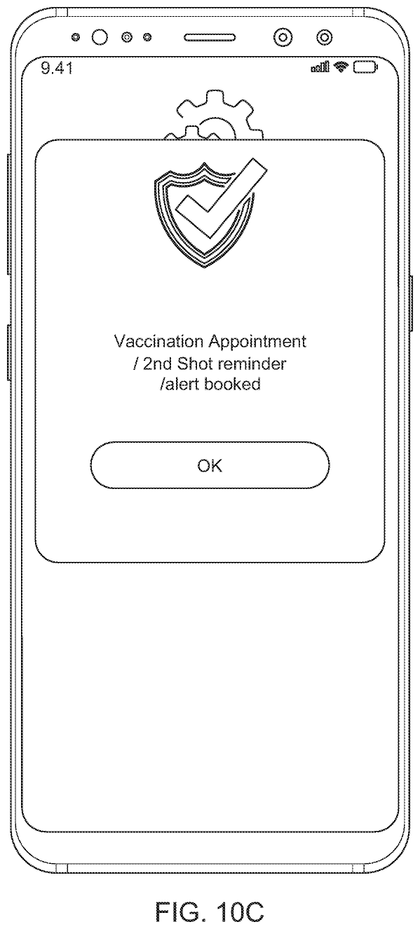

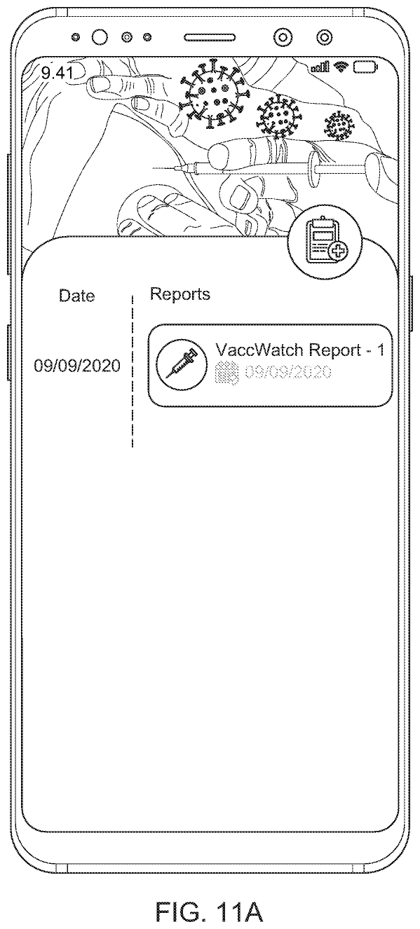

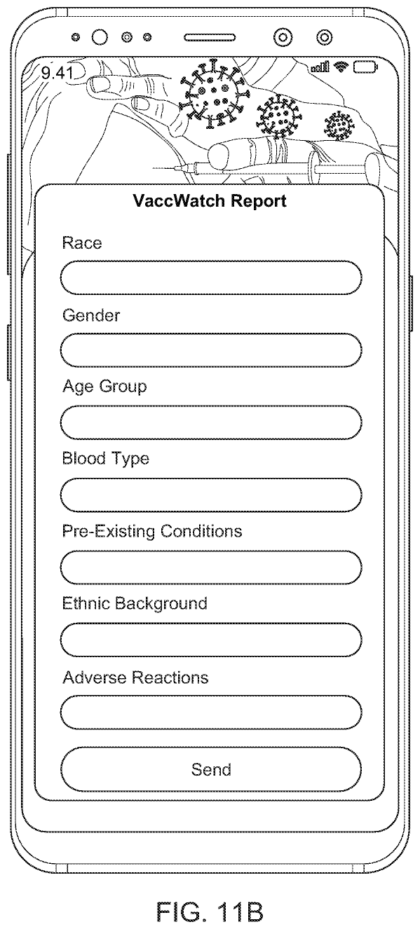

Referring now to FIGS. 1 through 11B, the present invention may include the following systemic interrelated systemic components:

Component 1: collection of vaccinated individual data embodied in a centralized inventory of all individuals to be vaccinated against any virus (or any pathogen), which may include a login/account interface provided through a user interface of a computer; the vaccinated individual data may include pre-existing vaccination records.

Component 2: validation of Medical Service Provider (MSP) credential data by way of inputting a National Provider Identifier (NPI No.), which is a 10-digit numerical identifier, or a DEA No. via the interface of the user interface.

Component 3: verification of vaccination administration; for instance, after a Component-2 validated MSP administers the one or two doses of the vaccine such administration may be verified by said validated MPS inputting their NPI No. or DEA No. through the computer user interface, wherein the present invention is configured to generate unique QR code or pass code; the QR code or pass code generator works individually but will also rely on vaccinated individual data validation of MSP credential data.

Component 4: vaccination data storage on a cloud database to avoid duplication and conduct an efficient and more streamlined vaccination campaign. Component 5: post-vaccination profile generation through QR code scanning, wherein the scanning may involve an image capturing device (e.g., camera) of the computer.

Component 6: is an alternative to Component 5, wherein the post-vaccination profile can be created via the systemic software application using a unique pass code along with one or more digital identification, wherein the digital identification can include a verified email, a verified mobile phone number, first and last name, city, state and country, blood type rhesus, or the like.

Component 7: generate an encrypted universal certificate subsequent establishment of the post-vaccination profile either by the input of a unique passcode or the scanning of the QR code.

Component 8: Issuance of a Vaccination Certificate is in effect the output of Component 7.

Component 9: display/verification of the issuance of the Vaccination Certificate, which may include display on an computer user interface, but may also include generation of an exportable facsimile, or conversion to an exportable electronic file, such as but not limited to a PDF to be emailed, a text messaged. Component 9 is dependent on Components 1 through 8.

The present invention may include an algorithmic generated vaccine/immunization list and, in certain embodiments, most specifically the COVID-19 vaccine manufacturers, based on birth date or vaccine start date and completion date, geographical areas, priority group such as medical professionals, immune compromised individuals, essential worker, teachers, etc. The present invention may include immunization tables for children, teen, adults, and special populations to visually determine necessary immunizations. The present invention includes information on all FDA-approved vaccines in the United States for COVID-19 virus and others including NDC No., dosage, frequency, and contraindications. The present invention may include information on disease states that can be prevented by vaccination, including information on the vaccine side effects and how it could be mitigated by not targeting or focusing on certain ethnic group of people, or certain individual from a geographical area.

The willingness to provide patient vaccination information to issue a vaccination certificate is vital, the unique QR code or pass code are also important to have in the application development. The verification of emails and phone number and potentially matching them to the name of the individual are features that can be added to enhance the application security and the data integrity.

The vaccination could be completed for certain individuals and the same people could later register with the application or web site entering all necessary information with the intent of requesting the Universal Vaccination Certificate. The post-vaccination registration will allow the present invention to be more inclusive and broaden its subscriber base and reach individuals in remote parts of the country or the world.

The present invention may be use to solve several problems: the main one may be to efficiently streamline the administration of a worldwide vaccination campaign, priorities certain groups of individuals that are either essential or immune compromised, government workers, medical professional, law enforcement members, etc.

The present invention may be used to solve a second problem: the record keeping of all individuals that were vaccinated, are in line to be vaccinated or refuse to be vaccinated.

The third problem the present invention may solve is once an individual has been vaccinated, how do we know he is immune and vaccinated and can no longer spread the virus and infect others? The issuance and usage of the secured and encrypted Universal Vaccination Certificate will do just that. It will serve as a universal tool to evidence the vaccination against a certain virus in a standard recognizable format. For example, if one wonders whether he can schedule a meeting with certain colleagues, parents being invited in their children school, flying on a commercial flight, inviting certain members at a gathering, year-end function or a wedding, the Universal Vaccination Certificate embodied in the present invention will be the tool to provide third parties the assurance that the one individual at question has a legitimate proof of vaccination that it is credible and has a universal format.

In certain embodiments the present invention provides a platform, which accounts, inventory within a centralize database of individuals to be vaccinated by validating the credential of Medical Service Providers (MSPs) administering the one or two doses of the vaccine through an NPI # or DEA # which are verified in real time.

A unique QR code or passcode may subsequently be generated to each vaccinated user which would be printed on the resulting certificate.

The present invention aims to efficiently streamline groups of individuals that need to be vaccinated in priority classes such as medical personal, elderly, immunocompromised individuals, government workers, members of the law enforcement community, etc.

The present invention may provide a cloud based universal platform for all relevant data pertaining to the individuals being vaccinated, and the issuance of a universal secured encrypted vaccination certificate, a database of self reported adverse reactions to vaccines. A QR code and passcode generator will be issued and licensed to MSPs, thereby facilitating an online vaccination certificate issuance function of the present invention and maximize what would have been many individual unrelated actions into a collective efficient database.

Each patient may register through the systemic software application using simple registration, Facebook, Twitter, or Google+ profile. The simple registration may be performed with a verified email account and a verified phone number with a six-digit numerical passcode being generated for enhanced security.

Individuals vaccinated, and their updated information, may be stored on a cloud database to avoid duplication and conduct an efficient and more streamlined vaccination campaign. Upon completion of the vaccination administration, a QR code shall be scanned or read with an iOS iPhone or Android phone that will issue an encrypted 128- or 256-bit certificate. Each certificate may be issued to a name and may provide blood type rhesus, date of birth, city, state and country of vaccination, the Issue date, the vaccine manufacturer related to said certified vaccination embodied in the universal vaccination certificate.

The issued universal vaccination certificate may then be displayed on the systemic software application, constituting a health credential and shown using a smart phone or Tablet or shall be converted to a PDF, JPEG file to be sent by email, text message (SMS), faxed or sent through WhatsApp™, etc.

The platform enabled by the present invention may host a function named VaccWatch which is a sophisticated side effects tracker functionality, wherein injected/vaccinated recipients will be able to self-report their side effects and detail any adverse reactions experienced in accordance with their race, gender, age group, ethnic background, location so as to translate the side effect information into an easy to read charts.

The present invention ensures the safe and secure data collection of individuals being vaccinated and the issuance of a secured universal vaccine certificate through providing a cloud-based universal platform for all relevant data pertaining to the individuals being vaccinated, and the issuance of a universal secured encrypted vaccination certificate (as currently there is no other application can technologically guide a MPS through administration of a vaccine with a QR code reading tool which would translate into the issuance of an encrypted Universal Vaccination Certificate.

The present invention provides, under one umbrella, a cloud-based database that will ensure MSPs log in all vaccine recipients in this single universal database, wherein all such recipients will be issued a unique QR code which would be used to issue an encrypted vaccination certificate.

Additionally, the present invention could be applied to fields other than the vaccination certificate issuance, for instance it could replace or create secured electronic cards or ID's such as certain law enforcement members, licensed professionals (accountants, lawyers, medical doctors etc.), or could be used to issue professional licenses, diplomas, or produce universally recognized secured certificates, ID's and professional credentials such as passes or badges.

It should be understood, of course, that the foregoing relates to exemplary embodiments of the invention and that modifications may be made without departing from the spirit and scope of the invention as set forth in the following claims.

What is claimed is:

1. A method for creating an electronically encrypted and exportable vaccination certificate, the method comprising:
validating, via a first processor, credentials of a medical service provider (MSP) through inputting one or more MSP data sets;
collecting, via a second processor, one or more patient data sets;
generating, via the second processor, an exportable identifier (EI);
inputting, by the MSP through the first processor, a vaccination data set associated with the one or more patient data sets; and
generating, by the second processor, an encrypted and exportable vaccination certificate (EEVC) when the second processor receives an indicator generated by the first processor of said inputting of the vaccination data.

2. The method of claim 1, wherein the EI is a quick response code (QRC) or passcode.

3. The method of claim 1, wherein the vaccination data includes at least one date of vaccination and at least one number of administrations of a vaccine.

4. The method of claim 1, wherein the vaccination data includes the one or more MSP data sets.

5. The method of claim 1, wherein the EEVC includes the EI.

6. The method of claim 1, wherein the EEVC includes the vaccination data.

7. The method of claim 1, wherein the EEVC includes the one or more patient data sets.

8. The method of claim 1, wherein the inputting of said vaccination data is conditioned of the MSP administrating a vaccine.

9. The method of claim 1, further comprising representing, on the second processor a side effect tracker functionality configured to collect a self-report of adverse reactions.

10. A method for creating an electronically encrypted and exportable vaccination certificate, the method comprising:
validating, via a first processor, credentials of a medical service provider (MSP) through inputting one or more MSP data sets;
collecting, via a second processor, one or more patient data sets;
generating, via the second processor, a quick response code (QRC);
inputting, by the MSP through the first processor, a vaccination data set associated with the one or more patient data sets, wherein the inputting of said vaccination data is conditioned of the MSP administrating a vaccine, wherein the vaccination data comprises:
at least one date of vaccination;
at least one number of administrations of the vaccine; and
the one or more MSP data sets;
generating, by the second processor, an encrypted and exportable vaccination certificate (EEVC) when the second processor receives an indicator generated by the first processor of said inputting of the vaccination data, wherein the EEVC comprises:
the QCR;
the vaccination data; and
the one or more patient data sets; and
representing, on the second processor a side effect tracker functionality configured to collect a self-report of adverse reactions.
